(19) United States
(12) Patent Application Publication
(10) Pub. No.: US 2026/0078013 A1
(43) Pub. Date: Mar. 19, 2026

(54) INTELLIGENT DECISION-MAKING AND CONTROL METHOD AND SYSTEM FOR MICROCHEMICAL REACTION IN STEPWISE SULFIDATION OF HIGH ARSENIC-CONTAIN ED WASTE ACID FROM COPPER SMELTER

(71) Applicant: Tongji University, Shanghai (CN)

(72) Inventors: Linhua JIANG, Shanghai (CN); Ning DUAN, Shanghai (CN)

(73) Assignee: Tongji University, Shanghai (CN)

(21) Appl. No.: 19/303,496

(22) Filed: Aug. 19, 2025

(30) Foreign Application Priority Data
Sep. 13, 2024 (CN) 202411280283.X

(51) Int. Cl. C01G 28/00; G16C 20/10

(52) U.S. Cl. CPC C01G 28/008; G16C 20/10

(57) ABSTRACT

Provided are an intelligent decision-making and control method and system for a microchemical reaction in stepwise sulfidation of a high arsenic-contained waste acid from copper smelter. Microchemical information from the real-time monitoring module is acquired by the intelligent decision-making module. A stage combination for a sulfidation reaction is determined based on a concentration of arsenic in raw waste acid. A total amount of arsenic and an amount of hydrogen sulfide to be added at a first stage are calculated based on a valence state and concentration of arsenic in and a flow rate of inflow at the first stage, and a total amount of arsenic and an amount of hydrogen sulfide to be added at a second stage are calculated based on a valence state and concentration of arsenic in and a flow rate of inflow at the second stage.

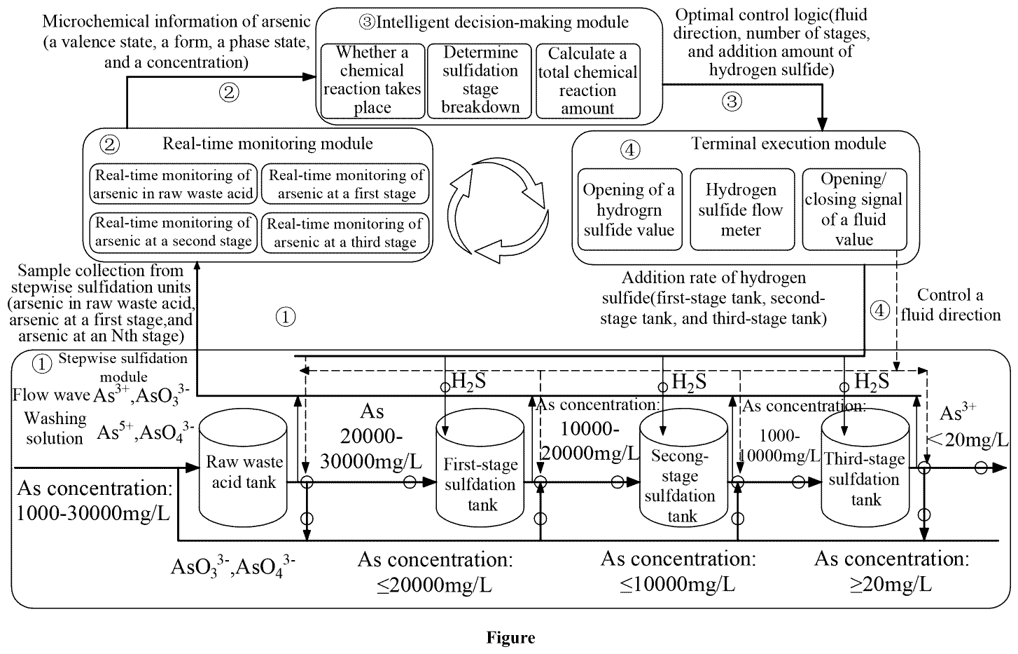

### CROSS REFERENCE TO THE RELATED APPLICATIONS

This application is based upon and claims priority to Chinese Patent Application No. 202411280283.X, filed on Sep. 13, 2024, the entire contents of which are incorporated herein by reference.

### TECHNICAL FIELD

The present disclosure relates to the technical field of clean waste acid treatment, and specifically relates to an intelligent decision-making and control method and system for a microchemical reaction in stepwise sulfidation of a high arsenic-contained waste acid from copper smelter.

### BACKGROUND

Currently, there is a large amount of waste acid resulting from copper pyrometallurgy in China, which reaches about 15,000,000 m³/year. Waste acid has a high heavy metal concentration. For example, an arsenic ion concentration in waste acid is as high as 30,000 mg/L. Arsenic exists in waste acid in complex and diverse forms, such as As³⁺, As⁵⁺, AsO₃³⁻, and AsO₄³⁻. There is a wide concentration range of arsenic in waste acid. For example, the maximum arsenic ion concentration is 300,000-fold different from the minimum arsenic ion concentration. As a result, waste acid poses a significant environmental risk. The precipitation technology for treating waste acid from copper smelter has advanced from lime precipitation to sulfide precipitation. Vulcanizing agents have also evolved from sodium sulfide and sodium hydrosulfide to hydrogen sulfide. However, according to the current practical operations, the problems, such as excessive arsenic in filtrates produced after sulfidation, large emissions of toxic gases, and excessive production of arsenic-contained hazardous waste, have not been solved significantly.

Most importantly, the existing analytical methods usually can only determine a total arsenic content in waste acid, and cannot analyze the actual valence states and forms of arsenic-contained species such as As³⁺, As⁵⁺, AsO₃³⁻, and AsO₄³⁻. Various units for a precipitation treatment process involve different chemical mechanisms, species forms, treatment concentrations, total substance amounts, and reaction efficiencies. Consequently, it is difficult for enterprises to accurately determine an amount of hydrogen sulfide to be added based on a valence state and concentration of a species in each unit, which leads to a high arsenic concentration in raw waste acid, a large amount of hydrogen sulfide added, and a high arsenic concentration in a filtrate at a production site, a high emission of toxic gases, and a large amount of hazardous waste generated. Therefore, the development of an intelligent decision-making and control method and system for a microchemical reaction in stepwise sulfidation of high arsenic-contained waste acid from copper smelter is an urgent need for enterprises to improve the production efficiency, reduce the high arsenic-contained hazardous waste, reduce the production cost, and enable the up-to-standard discharge.

### SUMMARY

An objective of the present disclosure is to provide an intelligent decision-making and control method and system for a microchemical reaction in stepwise sulfidation of a high arsenic-contained waste acid from copper smelter. Accordingly, the present disclosure solves the problem that, because it is difficult to accurately determine an amount of hydrogen sulfide to be added based on a valence state and concentration of a species in each unit, there is a high arsenic concentration in raw waste acid, a large amount of hydrogen sulfide added, and a high arsenic concentration in a filtrate at a production site, a high emission of toxic gases, and a large amount of hazardous waste generated.

To achieve the above objective, the present disclosure provides the following technical solutions:

In a first aspect, the present disclosure provides an intelligent decision-making and control system for a microchemical reaction in stepwise sulfidation of a high arsenic-contained waste acid from copper smelter, including a stepwise sulfidation module, a real-time monitoring module, an intelligent decision-making module, and a terminal execution module.

The stepwise sulfidation module comprising a plurality of containers, is configured to implement a sulfidation reaction; where the plurality of containers are divided into a plurality of gradient stages; the plurality of containers are connected through main pipes and bypass pipes; electromagnetic valves are installed on the main pipes and the bypass pipes to open or close a corresponding pipe; and a flow meter for flow monitoring is provided on each of the main pipes and the bypass pipes.

The real-time monitoring module is configured to allow sample collection from the plurality of containers, detect microchemical information of arsenic including a valence state, a form, a phase state, and a concentration of the arsenic, and transmit corresponding information to the intelligent decision-making module.

The intelligent decision-making module is configured to intelligently determine whether the sulfidation reaction is able to occur based on a form of arsenic in a fluid determined by the real-time monitoring module; configured to return the fluid to a previous procedure (In the previous procedure, acidity is adjusted to change arsenic from arsenite ion to arsenic cation) when the arsenic exists in a form of an anionic group and thus the sulfidation reaction fails to occur; and allow the fluid to enter the stepwise sulfidation module when the arsenic exists in a form of a cation and thus the sulfidation reaction is able to occur; and according to an arsenic ion concentration, split the fluid, and then allow split fluid to enter the plurality of containers at different gradient stages; according to an arsenic concentration and a flow rate of inflow at each stage, calculate a total amount of the arsenic; and according to a valence state of the arsenic and a molar ratio of a chemical reaction, automatically calculate a total amount of hydrogen sulfide to be added.

The terminal execution module is configured to: based on an instruction issued by the intelligent decision-making module, open or close electromagnetic valves on the main pipes and the bypass pipes to achieve automatic gradient stage switching among the plurality of containers, and adjust an opening degree of a valve on a hydrogen sulfide pipe to control a total amount and a rate of hydrogen sulfide to be added to a container in a staged reaction unit.

As an embodiment of the present disclosure, the plurality of containers in the stepwise sulfidation module include a raw waste acid tank for raw waste acid storage, a first-stage sulfidation tank for a first-stage treatment, a second-stage sulfidation tank for a second-stage treatment, and a third-stage sulfidation tank for a third-stage treatment.

As an embodiment of the present disclosure, a total content of arsenic introduced into the first-stage sulfidation tank is greater than or equal to 20,000 mg/L, a total content of arsenic introduced into the second-stage sulfidation tank is greater than or equal to 10,000 mg/L, and a total content of arsenic introduced into the third-stage sulfidation tank is greater than or equal to 20 mg/L.

As an embodiment of the present disclosure, the electromagnetic valves on the main pipes and the bypass pipes among the first-stage sulfidation tank, the second-stage sulfidation tank, and the third-stage sulfidation tank are opened or closed to enable stage switching or a multi-stage combination among the first-stage sulfidation tank, the second-stage sulfidation tank, and the third-stage sulfidation tank.

As an embodiment of the present disclosure, the real-time monitoring module includes an adjustable high-purity monochromatic light source, a multi-optical-path combined sample flow cell, a multi-point photodetector, and a weak signal processor; the multi-optical-path combined sample flow cell is connected to the plurality of containers in the stepwise sulfidation module; the adjustable high-purity monochromatic light source is configured to irradiate a fluid in the multi-optical-path combined sample flow cell, where after penetrating through the fluid, incident light undergoes an attenuation to produce transmitted light, and corresponding spectral signals are acquired through the multi-point photodetector and the weak signal processor.

As an embodiment of the present disclosure, the intelligent decision-making module is configured to calculate a total arsenic mass according to Q_{As}=arsenic concentration*fluid flow rate, and calculate a total hydrogen sulfide mass according to Q_{H2S}=K*96/150*Q_{As}, where the K is an excess coefficient.

As an embodiment of the present disclosure, the anionic group includes AsO₃³⁻ and AsO₄³⁻, the cation includes As³⁺ and As⁵⁺, and the valence state of arsenic includes As³⁺ and As⁵⁺; and a value of the K ranges from 1.1 to 1.6.

As an embodiment of the present disclosure, the main pipes and the bypass pipes in the stepwise sulfidation module have equal diameters, and an opening degree of each electromagnetic valve on the main pipes and the bypass pipes ranges from 0% to 100%.

In a second aspect, the present disclosure also provides an intelligent decision-making and control method for a microchemical reaction in stepwise sulfidation of a high arsenic-contained waste acid from copper smelter, where the method is applied to the above-mentioned system, and includes:
step S1, introducing an arsenic-contained waste acid fluid into the stepwise sulfidation module, and allowing the arsenic-contained waste acid fluid circulate within the plurality of containers in the stepwise sulfidation module;
step S2, when the arsenic-contained waste acid fluid is within the plurality of containers in the stepwise sulfidation module, detecting the valence state, the form, the phase state, and the concentration of arsenic in the arsenic-contained waste acid fluid by the real-time monitoring module;
step S3, transmitting monitored information to the intelligent decision-making module; when the form of the arsenic in the arsenic-contained waste acid fluid does not allow a sulfidation reaction, closing circulating electromagnetic valves in the stepwise sulfidation module to cause the arsenic-contained waste acid fluid to return; when the sulfidation reaction is able to occur, based on the concentration of the arsenic in the arsenic-contained waste acid fluid, opening circulating electromagnetic valves of the stepwise sulfidation module at corresponding sites, such that the plurality of containers in the stepwise sulfidation module are divided into a plurality of stages; and through the terminal execution module, controlling addition of a corresponding amount of a hydrogen sulfide gas to a container at a corresponding end in the stepwise sulfidation module, such that the arsenic in the arsenic-contained waste acid fluid undergoes the sulfidation reaction in the plurality of containers at the plurality of stages; and
step S4, when a concentration of arsenic in a filtrate produced after a waste acid treatment reaches a discharge standard, discharging the filtrate through an outlet pipe of the stepwise sulfidation module.

As an embodiment of the present disclosure, the concentration of the arsenic in the filtrate produced after the waste acid treatment is less than 20 mg/L.

Compared with the prior art, the present disclosure has the following beneficial effects:
1. In the present disclosure, microchemical information from the real-time monitoring module is acquired by the intelligent decision-making module. Whether a chemical reaction can occur is determined based on the analysis of a form and a phase state of arsenic in raw waste acid, and a stage combination for a sulfidation reaction is determined based on a concentration of arsenic in raw waste acid. A total amount of arsenic and an amount of hydrogen sulfide to be added at a first stage are calculated based on a valence state and concentration of arsenic in the first stage and a flow rate of inflow at the first stage, a total amount of arsenic and an amount of hydrogen sulfide to be added at a second stage are calculated based on a valence state and concentration of arsenic in the second stage and a flow rate of inflow at the second stage, and a total amount of arsenic and an amount of hydrogen sulfide to be added at a third stage are calculated based on a valence state and concentration of arsenic in the third stage and a flow rate of inflow at the third stage. Finally, a liquid splitting direction and an addition rate of hydrogen sulfide for a reaction at each stage are dynamically adjusted by the terminal execution module to achieve the closed-loop feedback and optimal control of the adjustment of a liquid flow direction based on a concentration and the accurate addition of hydrogen sulfide, which is conducive to improving the efficiency of sulfidation production and can reduce the generation of arsenic-contained hazardous waste.
2. In the present disclosure, the electromagnetic valves on the main pipes and the bypass pipes among the first-stage sulfidation tank, the second-stage sulfidation tank, and the third-stage sulfidation tank are opened or closed to enable the stage switching or the multi-stage combination among the first-stage sulfidation tank, the second-stage sulfidation tank, and the third-stage sulfidation tank. The terminal execution module can determine a concentration of arsenic in the current raw waste acid tank based on an arsenic content signal in waste acid transmitted by the real-time monitoring module. The electromagnetic valves on the plurality of main pipes and bypass pipes in the stepwise sulfidation module are opened or closed to enable the gradient stage switching among the first-stage sulfidation tank, the second-stage sulfidation tank, and the third-stage sulfidation tank at different gradient stages. Accordingly, the first-stage sulfidation tank, the second-stage sulfidation tank, and the third-stage sulfidation tank on multi-optical-path can achieve the multi-stage sulfidation treatment of waste acid fluids with varying concentrations and flow rates. Therefore, the present disclosure allows a variable treatment gradient and has a wide applicability range.

### BRIEF DESCRIPTION OF THE DRAWINGS

Figure is a general schematic diagram of the overall system of the present disclosure.

### DETAILED DESCRIPTION OF THE EMBODIMENTS

The technical solutions of the embodiments of the present disclosure are clearly and completely described below with reference to the accompanying drawings in the embodiments of the present disclosure. Apparently, the embodiments are merely some rather than all of the embodiments of the present disclosure. All other embodiments obtained by a person of ordinary skill in the art based on the embodiments of the present disclosure without creative efforts shall fall within the protection scope of the present disclosure.

### Example 1

Figure is a general schematic diagram of the overall system of the present disclosure. That is, an intelligent decision-making and control system for a microchemical reaction in stepwise sulfidation of a high arsenic-contained waste acid from copper smelter is provided, including a stepwise sulfidation module, a real-time monitoring module, an intelligent decision-making module, and a terminal execution module.

The stepwise sulfidation module including a plurality of containers, is configured to implement a sulfidation reaction, where the plurality of containers are divided into a plurality of gradient stages. The plurality of containers are connected through main pipes and bypass pipes. Electromagnetic valves are installed on the main pipes and bypass pipes to open or close a corresponding pipe. The plurality of containers are configured to store the waste acid fluid introduced and make the waste acid fluid circulate among the plurality of containers through the main pipes and the bypass pipes. A flow meter for flow monitoring is provided on each of the main pipes and the bypass pipes. A circulating flow rate among the plurality of containers in the stepwise sulfidation module is detected by the flow meter, and the detected information is transmitted to the real-time monitoring module, the intelligent decision-making module, and the terminal execution module. After a waste acid is introduced into a container at a corresponding gradient stage, an opening of a hydrogen sulfide valve is controlled by the terminal execution module to introduce hydrogen sulfide, and the hydrogen sulfide is allowed to react with As³⁺ and As⁵⁺ ions, thereby achieving the gradient sulfidation-based removal of arsenic.

The real-time monitoring module is configured to: allow sample collection from the plurality of containers, detect microchemical information of arsenic including a valence state, a form, a phase state, concentrations of arsenic at different valence states, and concentrations of arsenic in different forms, and transmit corresponding information to the intelligent decision-making module. Based on the detection results of valence states, forms, and phase states of arsenic in the plurality of containers, microchemical information including the valence state and the form (As³⁺, As⁵⁺, AsO₃³⁻, and AsO₄³⁻), and the concentration of arsenic in waste acid is uploaded into the intelligent decision-making module in real time.

The intelligent decision-making module is configured to intelligently determine whether the sulfidation reaction is able to occur based on a form of arsenic in a fluid determined by the real-time monitoring module. When the arsenic exists in a form of an anionic group (AsO₃³⁻ and AsO₄³⁻), the sulfidation reaction fails to occur, and the fluid is returned to a previous procedure. When the arsenic exists in a form of a cation (As³⁺ and As⁵⁺), the sulfidation reaction is able to occur, and the fluid is allowed to enter the stepwise sulfidation module. The fluid is split according to an arsenic ion concentration and then allowed to enter the plurality of containers at different gradient stages. According to an arsenic concentration and a flow rate of inflow at each stage, a total amount of arsenic is calculated. According to a valence state (As³⁺ and As⁵⁺) of arsenic and a molar ratio for a chemical reaction, a total amount and rate of hydrogen sulfide to be added are automatically calculated. The intelligent decision-making module is configured to, based on a thermodynamic principle (whether the sulfidation reaction can occur depends on a form of arsenic, in the case that the sulfidation reaction can occur 2As³⁺+3S²⁻→As₂S₃↓ (a solid precipitate) and 2As⁵⁺+5S²⁻→As₂S₃↓+2S↓ (a solid precipitate); and in the case that the sulfidation reaction cannot occur AsO₃³⁻+S²⁻ and AsO₄³⁻+S²⁻ cannot lead to a precipitate), intelligently determine whether the sulfidation reaction can take place according to the form of arsenic in raw waste acid determined by the real-time monitoring module. If arsenic is AsO₃³⁻ or AsO₄³⁻, the sulfidation reaction cannot take place, and the raw waste acid is returned to a previous procedure and is strictly prohibited from entering a sulfidation unit, where if arsenic is As³⁺ or As⁵⁺, the sulfidation reaction can take place, and the raw waste acid is allowed to enter sulfidation process. The intelligent decision-making module is further configured to, by using a kinetic principle (an extent of the sulfidation reaction depends on a valence state of arsenic 2As³⁺ (relative atomic mass: 150)+3S²⁻(relative atomic mass: 96)→As₂S₃↓ and 2As⁵⁺ (relative atomic mass: 150)+5S²⁻ (relative atomic mass: 160)→As₂S₃↓+2S↓, and As⁵⁺ consumes 60% more sulfur than As³⁺), intelligently determine the number of sulfidation stages based on an arsenic concentration provided by the real-time monitoring module. The intelligent decision-making module is further more configured to, according to the chemical reaction equation: 2As³⁺+3S²⁻=As₂S₃ and further a valence state of arsenic provided by the real-time monitoring module, send optimal control logic information to the terminal execution module.

The terminal execution module is configured to: open or close electromagnetic valves on the main pipes and the bypass pipes based on an instruction issued by the intelligent decision-making module to achieve automatic gradient stage switching among the plurality of containers, and adjust an opening degree of a valve on a hydrogen sulfide pipe to control a total amount and a rate of hydrogen sulfide to be added to a container in a staged reaction unit. The terminal execution module is configured: 1) to control an outflow direction of the raw waste acid and the opening or closing of a corresponding electromagnetic valve based on a form of arsenic in the raw waste acid, for example, if a form of arsenic is AsO₃³⁻ or AsO₄³⁻, a return pipe valve is opened and a sulfidation pipe valve is closed, and if a form of arsenic is As³⁺ or As⁵⁺, the return pipe valve is closed and the sulfidation pipe valve is opened; 2) automatically adjust the opening or closing of valves on the main pipes and the bypass pipes for raw waste acid fluids based on a concentration of arsenic in raw waste acid; and 3) according to a pressure in a hydrogen sulfide tank and transient data from a hydrogen sulfide flow meter, dynamically adjust an opening of the hydrogen sulfide valve to meet the requirements for an amount and a rate of hydrogen sulfide to be added to the staged reaction unit.. Based on thermodynamic and kinetic reaction principles, microchemical information about arsenic in a staged tank is acquired by the real-time monitoring module and fed back to the intelligent decision-making module. The intelligent decision-making module determines whether a sulfidation reaction can occur based on a form of arsenic in raw waste acid, determines a molar ratio for the sulfidation reaction which uses hydrogen sulfide as the raw material based on a valence state of arsenic, determines the number of sulfidation reaction stages based on a concentration of arsenic, and calculates a total amount of arsenic and an amount of hydrogen sulfide to be added based on a concentration and flow rate of an influent liquid. Finally, a liquid splitting direction and a hydrogen sulfide addition rate are dynamically adjusted by the terminal execution module to achieve the closed-loop feedback and optimal control of the adjustment of a liquid splitting direction based on a concentration and the accurate addition of hydrogen sulfide during a sulfidation process. The above technology can significantly improve the production efficiency, reduce the generation of arsenic-contained hazardous waste, reduce the production cost, and meet the up-to-standard discharge requirement.

As shown in Figure, the real-time monitoring module includes an adjustable high-purity monochromatic light source, a multi-optical-path combined sample flow cell, a multi-point photodetector, and a weak signal processor. The multi-optical-path combined sample flow cell is connected to the plurality of containers in the stepwise sulfidation module. The adjustable high-purity monochromatic light source is configured to irradiate a fluid in the multi-optical-path combined sample flow cell, where after penetrating through the fluid, incident light undergoes an attenuation to produce transmitted light. Corresponding spectral signals are acquired through the multi-point photodetector and the weak signal processor. The microchemical information mainly refers to an occurrence existential state of arsenic, such as a trivalent arsenic cation As³⁺, a pentavalent arsenic cation As⁵⁺, an arsenite ion AsO₃³⁻, and an arsenate ion AsO₄³⁻, and a corresponding concentration C thereof.

As shown in Figure, the intelligent decision-making module is configured to calculate a total arsenic mass according to Q_{As}=arsenic concentration*fluid flow rate, and calculate a total hydrogen sulfide mass according to Q_{H2S}=K*96/150 * Q_{As}, where K is an excess coefficient. Data on the trivalent arsenic cation As³⁺ and the pentavalent arsenic cation As⁵⁺ that is acquired by the real-time monitoring module is transmitted to the terminal execution module, and fed back to the intelligent decision-making module (hardware includes a server and a computer, and software includes a logical decision-making model and a chemical calculation model). A calculated hydrogen sulfide amount is transmitted to the terminal execution module, such that the terminal execution module can send the optimal control logic information to a terminal actuator. The hydrogen sulfide amount is multiplied by the excess coefficient K. K ranges from 1.1 to 1.6, which ensures that arsenic ions can react with hydrogen sulfide to produce a precipitate.

As shown in Figure, the main pipes and the bypass pipes in the stepwise sulfidation module have equal diameters, and an opening of each electromagnetic valve on the main pipes and the bypass pipes ranges from 0% to 100%. The rapid transport of a fluid through the main pipes and the bypass pipes with equal diameters ensures the timely and rapid flow of the fluid. The electromagnetic valves on the main pipes and the bypass pipes can be quickly opened or closed.

### Example 2

As shown in Figure, the plurality of containers in the stepwise sulfidation module include a raw waste acid tank for raw waste acid storage, a first-stage sulfidation tank for a first-stage treatment, a second-stage sulfidation tank for a second-stage treatment, and a third-stage sulfidation tank for a third-stage treatment. An arsenic concentration of a waste acid in the raw waste acid tank is detected. Based on a concentration gradient change of arsenic, the waste acid is introduced into the first-stage sulfidation tank at the corresponding stage through the main pipes and the bypass pipes for a sulfidation reaction and electromagnetic valves, and subjected to a sulfidation treatment with a hydrogen sulfide gas introduced. After an arsenic concentration gradient decreases, a resulting waste acid is then introduced into the second-stage sulfidation tank and the third-stage sulfidation tank successively. The above sulfidation treatment steps are repeated to carry out a three-stage stepwise sulfidation reaction, which controls a reaction gradient reasonably and can effectively treat waste acid resulting from the daily copper pyrometallurgy.

As shown in Figure, a total content of arsenic introduced into the first-stage sulfidation tank is greater than or equal to 20,000 mg/L, a total content of arsenic introduced into the second-stage sulfidation tank is greater than or equal to 10,000 mg/L, and a total content of arsenic introduced into the third-stage sulfidation tank is greater than or equal to 20 mg/L. If an arsenic content is 20,000 mg/L to 30,000 mg/L, it is classified as third-stage sulfidation. If an arsenic content is 10,000 mg/L to 20,000 mg/L, it is classified as second-stage sulfidation. If an arsenic content is 20 mg/L to 10,000 mg/L, it is classified as a first-stage sulfidation. As a result, sulfidation treatments at different gradient concentrations are carried out. Different arsenic concentrations correspond to different rates for introducing a hydrogen sulfide gas, which facilitates the terminal execution module to issue a corresponding instruction, strictly controls the amount of hydrogen sulfide introduced, and reduces the raw material waste while making a waste acid treatment meet a standard.

Further, the electromagnetic valves on the main pipes and the bypass pipes among the first-stage sulfidation tank, the second-stage sulfidation tank, and the third-stage sulfidation tank are opened or closed to enable the stage switching or the multi-stage combination among the first-stage sulfidation tank, the second-stage sulfidation tank, and the third-stage sulfidation tank. The terminal execution module can determine a concentration of arsenic in the current raw waste acid tank based on an arsenic content signal in waste acid transmitted by the real-time monitoring module. The electromagnetic valves on the plurality of main pipes and bypass pipes in the stepwise sulfidation module are opened or closed to enable the gradient stage switching among the first-stage sulfidation tank, the second-stage sulfidation tank, and the third-stage sulfidation tank at different gradient stages, thereby achieving the automatic switching of first-stage sulfidation→second-stage sulfidation→third-stage sulfidation. Accordingly, the first-stage sulfidation tank, the second-stage sulfidation tank, and the third-stage sulfidation tank each with multiple paths can achieve the multi-stage sulfidation treatment of waste acid fluids with varying flow rates. Therefore, the present disclosure allows a variable treatment gradient, has a wide applicability range, and enables a high sulfidation treatment efficiency for waste acid.

An intelligent decision-making and control method for a microchemical reaction in stepwise sulfidation of a high arsenic-contained waste acid from copper smelter is provided. The method is applied to the above system, and includes steps S1-S4.

In step S1, an arsenic-contained waste acid fluid is introduced into the stepwise sulfidation module, and allowed to circulate within the plurality of containers in the stepwise sulfidation module.

In step S2, when the arsenic-contained waste acid fluid is in the plurality of containers in the stepwise sulfidation module, the valence state, the form, the phase state, and the concentration of arsenic in the arsenic-contained waste acid fluid are detected by the real-time monitoring module.

In step S3, monitored information is transmitted to the intelligent decision-making module. When the form of the arsenic in the arsenic-contained waste acid fluid does not allow a sulfidation reaction, circulating electromagnetic valves in the stepwise sulfidation module are closed to cause the arsenic-contained waste acid fluid to return. When the sulfidation reaction can occur, based on the concentration of the arsenic in the arsenic-contained waste acid fluid, circulating electromagnetic valves of the stepwise sulfidation module at corresponding sites are controlled to open, such that the plurality of containers in the stepwise sulfidation module are divided into a plurality of stages. Through the terminal execution module, addition of a corresponding amount of a hydrogen sulfide gas is controlled to a container at a corresponding end in the stepwise sulfidation module, such that the arsenic in the arsenic-contained waste acid fluid undergoes the sulfidation reaction in the plurality of containers at the plurality of stages.

In step S4, when a concentration of arsenic in a filtrate produced after a waste acid treatment reaches a discharge standard, the filtrate is discharged through an outlet pipe of the stepwise sulfidation module.

As shown in Figure, the concentration of the arsenic in the filtrate produced after the waste acid treatment is less than 20 mg/L, indicating that the multi-stage gradient sulfidation treatment meets a discharge standard.

The above are merely preferred specific embodiments of the present disclosure, but the protection scope of the present disclosure is not limited thereto. Any equivalent replacement or modification made by a person skilled in the art according to the technical solutions of the present disclosure and the inventive concepts thereof within the technical scope of the present disclosure shall fall within the protection scope of the present disclosure.

What is claimed is:

1. An intelligent decision-making and control system for a microchemical reaction in stepwise sulfidation of a high arsenic-contained waste acid from copper smelter, comprising a stepwise sulfidation module, a real-time monitoring module, an intelligent decision-making module, and a terminal execution module, wherein
the stepwise sulfidation module comprises a plurality of containers and is configured to implement a sulfidation reaction, wherein the plurality of containers are divided into a plurality of gradient stages; the plurality of containers are connected through main pipes and bypass pipes; electromagnetic valves are installed on the main pipes and the bypass pipes to open or close a corresponding pipe; and a flow meter for flow monitoring is provided on each of the main pipes and the bypass pipes; the plurality of containers in the stepwise sulfidation module comprise a raw waste acid tank for raw waste acid storage, a first-stage sulfidation tank for a first-stage treatment, a second-stage sulfidation tank for a second-stage treatment, and a third-stage sulfidation tank for a third-stage treatment; and the electromagnetic valves on the main pipes and the bypass pipes among the first-stage sulfidation tank, the second-stage sulfidation tank, and the third-stage sulfidation tank are opened or closed to enable stage switching or a multi-stage combination among the first-stage sulfidation tank, the second-stage sulfidation tank, and the third-stage sulfidation tank;
the real-time monitoring module is configured to:
allow sample collection from the plurality of containers, detect microchemical information of arsenic comprising detection and analysis of a valence state, a form, a phase state, and a concentration of the arsenic, and transmit corresponding information to the intelligent decision-making module, wherein
the real-time monitoring module comprises an adjustable high-purity monochromatic light source, a multi-optical-path combined sample flow cell, a multi-point photodetector, and a weak signal processor; the multi-optical-path combined sample flow cell is connected to the plurality of containers in the stepwise sulfidation module; the adjustable high-purity monochromatic light source is configured to irradiate a fluid in the multi-optical-path combined sample flow cell, wherein after penetrating through the fluid, an incident light undergoes an attenuation to produce a transmitted light; and corresponding spectral signals are acquired through the multi-point photodetector and the weak signal processor;
the intelligent decision-making module is configured to:
intelligently determine whether the sulfidation reaction is allowed to occur based on a form of arsenic in the fluid determined by the real-time monitoring module;
return the fluid to a previous procedure when the arsenic exists in a form of an anionic group and thus the sulfidation reaction fails to occur;
allow the fluid to enter the stepwise sulfidation module when the arsenic exists in a form of a cation and thus the sulfidation reaction is allowed to occur;
split the fluid according to an arsenic ion concentration and then allow a split fluid to enter the plurality of containers at different gradient stages;
according to an arsenic concentration and a flow rate of inflow at each stage, calculate a total amount of the arsenic; and
according to a valence state of the arsenic and a molar ratio for a chemical reaction, automatically calculate a total amount of hydrogen sulfide to be added;
wherein the intelligent decision-making module is configured to calculate a total arsenic mass according to Q_{As}=arsenic concentration*fluid flow rate and a total hydrogen sulfide mass according to Q_{H2S}=K*96/150*Q_{As}, wherein the K is an excess coefficient; and
the terminal execution module is configured to:
open or close the electromagnetic valves on the main pipes and the bypass pipes based on an instruction issued by the intelligent decision-making module to achieve automatic gradient stage switching among the plurality of containers, and adjust an opening degree of a valve on a hydrogen sulfide pipe to control a total amount and a rate of hydrogen sulfide to be added to a container in a staged reaction unit.

2. The intelligent decision-making and control system for the microchemical reaction in stepwise sulfidation of the high arsenic-contained waste acid from copper smelter according to claim 1, wherein the anionic group comprises AsO₃³⁻ and AsO₄³⁻, the cation comprises As³⁺ and As⁵⁺, and the valence state of arsenic comprises As³⁺ and As⁵⁺; and a value of the K ranges from 1.1 to 1.6.

3. The intelligent decision-making and control system for the microchemical reaction in stepwise sulfidation of the high arsenic-contained waste acid from copper smelter according to claim 1, wherein the main pipes and the bypass pipes in the stepwise sulfidation module have equal diameters, and an opening degree of each of the electromagnetic valves on the main pipes and the bypass pipes ranges from 0% to 100%.

4. An intelligent decision-making and control method for a microchemical reaction in stepwise sulfidation of a high arsenic-contained waste acid from copper smelter, adopting the intelligent decision-making and control system for the microchemical reaction in stepwise sulfidation of the high arsenic-contained waste acid from copper smelter according to claim 1, and comprising following steps:
step S1, introducing an arsenic-contained waste acid fluid into the stepwise sulfidation module, and allowing the arsenic-contained waste acid fluid to circulate within the plurality of containers in the stepwise sulfidation module;
step S2, when the arsenic-contained waste acid fluid is within the plurality of containers in the stepwise sulfidation module, detecting the valence state, the form, the phase state, and the concentration of the arsenic in the arsenic-contained waste acid fluid by the real-time monitoring module;
step S3, transmitting monitored information to the intelligent decision-making module; when the form of the arsenic in the arsenic-contained waste acid fluid does not allow the sulfidation reaction, closing circulating electromagnetic valves in the stepwise sulfidation module to allow the arsenic-contained waste acid fluid to return; when the sulfidation reaction is allowed to occur, based on the concentration of the arsenic in the arsenic-contained waste acid fluid, opening the circulating electromagnetic valves of the stepwise sulfidation module at corresponding sites, such that the plurality of containers in the stepwise sulfidation module are divided into the plurality of gradient stages; and through the terminal execution module, controlling addition of a corresponding amount of a hydrogen sulfide gas to a container at a corresponding end in the stepwise sulfidation module, such that the arsenic in the arsenic-contained waste acid fluid undergoes the sulfidation reaction in the plurality of containers at the plurality of gradient stages; and
step S4, when a concentration of arsenic in a filtrate produced after a waste acid treatment reaches a discharge standard, discharging the filtrate through an outlet pipe of the stepwise sulfidation module.

5. The intelligent decision-making and control method for the microchemical reaction in stepwise sulfidation of the high arsenic-contained waste acid from copper smelter according to claim 4, wherein the anionic group comprises AsO₃³⁻ and AsO₄³⁻, the cation comprises As³⁺ and As⁵⁺, and the valence state of arsenic comprises As³⁺ and As⁵⁺; and a value of the K ranges from 1.1 to 1.6.

6. The intelligent decision-making and control method for the microchemical reaction in stepwise sulfidation of the high arsenic-contained waste acid from copper smelter according to claim 4, wherein the main pipes and the bypass pipes in the stepwise sulfidation module have equal diameters, and an opening degree of each of the electromagnetic valves on the main pipes and the bypass pipes ranges from 0% to 100%.

7. The intelligent decision-making and control method for the microchemical reaction in stepwise sulfidation of the high arsenic-contained waste acid from copper smelter according to claim 4, wherein the concentration of the arsenic in the filtrate produced after the waste acid treatment is less than 20 mg/L.
